(19) United States
(12) Patent Application Publication
(10) Pub. No.: US 2007/0272241 A1
(43) Pub. Date: Nov. 29, 2007

(54) System and Method for Scheduling Pause Maneuvers Used for Estimating Elastance and/or Resistance During Breathing

(76) Inventors: Warren G. Sanborn, Escondido, CA (US); Fernando Isaza, Carlsbad, CA (US); Francisco Lopez, Carlsbad, CA (US)

Correspondence Address: BAKER BOTTS L.L.P.;PATENT DEPARTMENT, 98 SAN JACINTO BLVD., SUITE 1500, AUSTIN, TX, 78701-4039, US

(21) Appl. No.: 11/383,128

(22) Filed: May 12, 2006

(51) Int. Cl. A61M 16/00

(52) U.S. Cl. 128/204.23

(57) ABSTRACT

A method of providing breathing assistance is provided. A plurality of a patient's breaths are assisted using a breathing assistance system. The plurality of breaths may include one or more pause breaths and one or more non-pause breaths, and the occurrence of pause breaths during the plurality of breaths may be randomized. Each pause breath may include a pause maneuver during which one or more valves of the breathing assistance system are closed to create a constant volume defined at least by the patient's lungs and one or more components of the breathing assistance system. One or more measurements may be taken during or proximate the pause maneuver, and one or more patient characteristic values may be determined based at least on the one or more measurements. The one or more patient characteristic values may include values for at least one of an elastance and a compliance associated with the patient.

### TECHNICAL FIELD

The present disclosure relates generally to the field of respiratory support, and more particularly to a system and method for scheduling pause maneuvers used for estimating a patient's elastance, compliance, and/or resistance during breathing.

### BACKGROUND

In a proportional assisted ventilation (PAV) system, a patient may be supplied with continuous assistance throughout an inspiratory effort in proportion to the moment-to-moment inspiratory effort provided by the patient, according to a PAV algorithm. Typically, none of the instantaneous inspiratory pressure, the instantaneous flow, or the resulting volume are set by the caregiver. The PAV breathing algorithm harmoniously links the ventilator to the patient, and the patient effectively “drives” the ventilator.

Values of the patient's lung-thorax elastance (or compliance) and lung resistance may be continuously or periodically estimated and inserted into the PAV breathing algorithm in order for the algorithm to function properly. These estimates may be calculated automatically by the ventilator and fed back into the PAV breathing algorithm such that the algorithm may adjust the breathing support supplied by the ventilator over time, as appropriate.

Elastance may generally be defined in terms of the elastic properties of the lungs and thorax, or the forces associated with expanding the lungs. In particular, the degree of stiffness of the lung-thorax region may be referred to as the elastance of the respiratory system. The elastance of the respiratory system may also be discussed in terms of compliance, which may be defined as the inverse of elastance. Generally, the easier it is to stretch the lung-thorax region, the lower the elastance (i.e., the greater the compliance).

Resistance forces, or the non-elastic forces at work in the breathing cycle, are the forces associated with moving air through a patient's airways. Lung resistance may be at least partially defined by a patient's physiological conditions. For example, patients suffering from asthma typically experience muscular constriction of the bronchi. Such patients may also experience swelling of the bronchial mucosa. The resistance, and thus the work required to achieve a particular amount of air flow through the breathing passageways, generally increases in proportion to the severity of such constriction. In some ventilation systems, flow and pressure sensors are used collect data for computing estimates of the patient's elastance (or compliance) and resistance.

The lung-thorax elastance and/or resistance of a patient may be determined or estimated in various manners, including using either direct or indirect approaches and following known algorithms. Some techniques for determining or estimating lung-thorax elastance and/or resistance utilize a relatively brief (e.g., 200-400 ms) pause maneuver at the end of inspiration, during which the ventilator system's inspiratory and expiratory valves are closed, thus establishing a closed volume including the patient's lungs and the breathing circuit. During this brief pause, the recoil pressure in the elastic lung-thorax may at least substantially equilibrate with the pressure trapped in the breathing circuit. The equilibrium pressure and/or the insufflation volume of the closed volume may be measured and used to estimate the patient's elastance (or compliance).

### SUMMARY

In accordance with the present disclosure, systems and methods for scheduling pauses used for estimating a patient's elastance, compliance, and/or resistance during breathing are provided.

According to one embodiment, a method of providing breathing assistance is provided. A plurality of a patient's breaths are assisted using a breathing assistance system. The plurality of breaths may include one or more pause breaths and one or more non-pause breaths, and the occurrence of pause breaths during the plurality of breaths may be randomized. Each pause breath may include a pause maneuver during which one or more valves of the breathing assistance system are closed to create a constant volume defined at least by the patient's lungs and one or more components of the breathing assistance system. One or more measurements may be taken during or proximate the pause maneuver, and one or more patient characteristic values may be determined based at least on the one or more measurements. The one or more patient characteristic values may include values for at least one of an elastance and a compliance associated with the patient.

According to another embodiment, another method of providing breathing assistance is provided. Multiple series of a patient's breaths may be assisted using a breathing assistance system, wherein each series of breaths includes a pause breath and a randomized number of non-pause breaths. Each pause breath may include a pause maneuver during which one or more valves of the breathing assistance system are closed to create a constant volume defined at least by the patient's lungs and one or more components of the breathing assistance system. Each non-pause breath may not include the pause maneuver.

According to another embodiment, a system for providing breathing assistance is provided. The system may include a breathing assistance controller, a scheduling module, a pause maneuver controller, one or more measurement devices, and a patient characteristic calculation module. The breathing assistance controller may be configured to assist a plurality of breaths for a patient, the plurality of breaths including one or more pause breaths and one or more non-pause breaths. The scheduling module may be configured to randomize the occurrence of pause breaths among the plurality of breaths. The pause maneuver controller may be configured to include a pause maneuver in each pause breath, the pause maneuver including closing one or more valves of the breathing assistance system to create a constant volume defined at least by the patient's lungs and one or more components of the breathing assistance system. The one or more measurement devices may be configured to take one or more measurements during or proximate the pause maneuver. The patient characteristic calculation module may be configured to calculate one or more patient characteristic values based at least on the one or more measurements, the one or more patient characteristic values including values for at least one of an elastance and a compliance associated with the patient.

According to another embodiment, a system for providing breathing assistance is provided. The system may include breathing assistance control means, pause scheduling means, pause controlling means, measuring means, and patient characteristic calculation means. The breathing assistance control means may assist a plurality of breaths for a patient, the plurality of breaths including one or more pause breaths and one or more non-pause breaths. The pause scheduling means may randomize the occurrence of pause breaths among the plurality of breaths. The pause controlling means may include a pause maneuver in each pause breath, the pause maneuver including closing one or more valves of the breathing assistance system to create a constant volume defined at least by the patient's lungs and one or more components of the breathing assistance system. The measuring means may take one or more measurements during or proximate the pause maneuver. The patient characteristic calculation means may calculate one or more patient characteristic values based at least on the one or more measurements, the one or more patient characteristic values including values for at least one of an elastance and a compliance associated with the patient.

According to another embodiment, a computer-readable medium including computer-executable instructions for providing breathing assistance may be provided. The instructions may include instructions for assisting a plurality of breaths for a patient using a breathing assistance system, the plurality of breaths including one or more pause breaths and one or more non-pause breaths. The occurrence of pause breaths during the plurality of breaths may be randomized. The instructions may further include instructions for including a pause maneuver in each pause breath during which one or more valves of the breathing assistance system are closed to create a constant volume defined at least by the patient's lungs and one or more components of the breathing assistance system. The instructions may further include instructions for taking or receiving one or more measurements during or proximate the pause maneuver, and instructions for determining values for at least one of an elastance and a compliance associated with the patient based at least on the one or more measurements.

### BRIEF DESCRIPTION OF THE DRAWINGS

Some embodiments of the disclosure may be understood by referring, in part, to the following description and the accompanying drawings, in which like reference numbers refer to the same or like parts, and wherein:

FIG. 1 illustrates a breathing assistance system for providing breathing assistance to a patient, according to one embodiment of the disclosure;

FIG. 2 illustrates an example gas delivery control system including a proportional assist ventilation (PAV) control system, according to one embodiment of the disclosure;

FIG. 3 illustrates an example method of using randomized pause maneuvers for determining at least one of the elastance and the compliance associated with a patient, according to one embodiment of the disclosure;

FIG. 4 illustrates another example method of using randomized pause maneuvers for determining at least one of the elastance and the compliance associated with a patient, according to one embodiment of the disclosure; and

FIG. 5 illustrates an example method of using randomized pause maneuvers for providing proportional assist ventilation (PAV), according to one embodiment of the disclosure.

### DETAILED DESCRIPTION

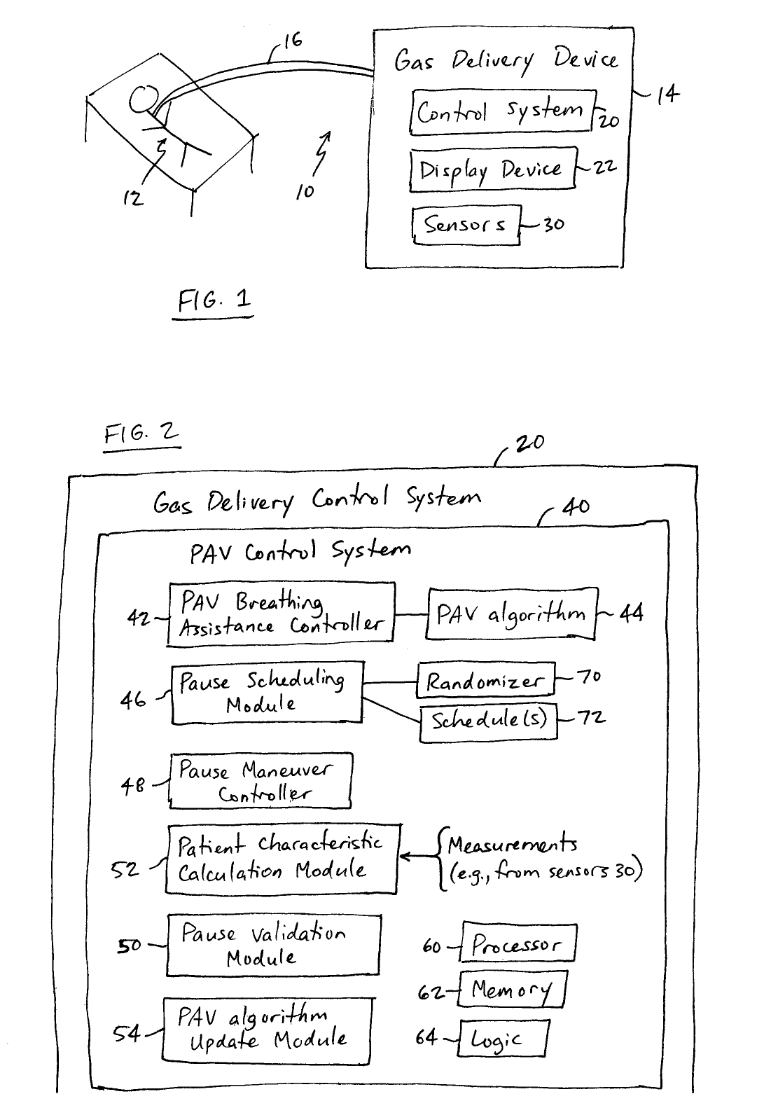

Selected embodiments of the disclosure may be understood by reference, in part, to FIGS. 1-5, wherein like numbers refer to same and like parts.

FIG. 1 illustrates an example breathing assistance system 10 for providing breathing assistance to a patient 12, according to one embodiment of the disclosure. Breathing assistance system 10 may include a gas delivery device 14, a patient circuit 16, and/or any other suitable systems or devices. In some embodiments, gas delivery device 14 may comprise a ventilator. For convenience, gas delivery device 14 is referred to herein as ventilator 14. However, is should be understood that gas delivery device 14 may include any other device used to deliver breathing gas or otherwise provide breathing assistance to a patient. Breathing gas may include, e.g., air, oxygen, and any one or more other gasses that may be delivered to a patient.

As used throughout this document, the term “ventilator” may refer to any device, apparatus, or system for delivering breathing gas to a patient, e.g., a ventilator, a respirator, a CPAP device, or a BiPAP device. The term “patient” may refer to any person or animal that is receiving breathing support from a ventilation system, regardless of the medical status, official patient status, physical location, or any other characteristic of the person. Thus, for example, patients may include persons under official medical care (e.g., hospital patients), persons not under official medical care, persons receiving care at a medical care facility, persons receiving home care, etc.

Ventilator 14 may include a gas delivery control system 20, one or more display devices 22, one or more sensors 30, and/or any other suitable components. Gas delivery control system 20 may be operable to control the ventilation support provided by ventilator 14 based on various inputs, e.g., inputs received from an operator and/or data received from various sensors 30.

Display devices 22 may be operable to display various data regarding the patient 12, the operation of ventilator 14, the ventilation of patient 14, and/or any other relevant data. In some embodiments, display device 22 may be fully or partially integrated with ventilator 14 and may comprise a touch screen display or other visual display. Display device 22 may be part of or otherwise associated with, a graphic user interface, which may be configured to display various information via display device 22 and/or provide an interface (e.g., a touch screen) for accepting input from human operators via display device 22 and/or other input devices (e.g., to set or modify ventilation settings, to access data, and/or to change or configure the display).

Patient circuit 16 may include any suitable means for connecting patient 12 to ventilator 14. For example, patient circuit 16 may comprise a breathing circuit including an inspiration conduit, an exhalation conduit, and/or a patient connection apparatus. The patient connection apparatus may include any device or devices configured to connect the breathing circuit to one or more breathing passageways of patient 12. For example, the patient connection apparatus may include a patient connection tube directly connected to the patient's trachea, an artificial airway (e.g., an endotracheal tube or other device) inserted in the patient's trachea, and/or a mask or nasal pillows positioned over the patient's nose and/or mouth. In embodiments including a patient connection tube, the patient connection tube may include a Wye (or “Y”) connector.

Breathing assistance system 10 may include one or more sensors 30 for sensing, detecting, and/or monitoring one or more parameters related to the ventilation of patient 12, e.g., parameters regarding the ventilation provided by ventilator 14 and/or physiological parameters regarding patient 12. For example, sensors 30 may include one or more devices for measuring various parameters of gas flowing into or out of patient 12 or ventilator 14, e.g., the pressure, flow rate, flow volume, temperature, gas content, and/or humidity of such gas flow. Thus, sensors 30 may include, e.g., one or more pressure sensors, flow meters, transducers, and/or oxygen sensors. Sensors 30 may be located at one or more various locations in breathing assistance system 10 for monitoring the pressure and or flow of gasses flowing into and/or out of patient 12 and/or ventilator 14. For example, one or more sensors 30 may be located in or proximate ventilator 14 and/or patient circuit 16. For example, depending on the particular embodiment, one or more sensors 30 may be located within or proximate to ventilator 14, an inspiration conduit and/or exhalation conduit of a patient circuit, an artificial airway, and/or a Wye connector.

As discussed above, gas delivery control system 20 may be operable to control the ventilation support provided by ventilator 14 based on various input received from an operator (e.g., via a touch screen and/or other user interfaces provided by ventilator 14) and/or data received from one or more sensors 30. For example, gas delivery control system 20 may regulate the pressure and/or flow of breathing gas delivered to a patient based at least on data received from sensors 30. Gas delivery control system 20 may include, or have access to, one or more processors, memory devices, and any other suitable hardware or software. The one or more memory devices may store instructions (e.g., any suitable software, algorithms, or other logic or instructions that may be executed by one or more processors) for controlling the operation of ventilator 14, e.g., controlling the ventilation support provided by ventilator 14.

In some embodiments, gas delivery control system 20 may be operable (in one or more ventilation modes) to automatically control (e.g., update or adjust) various ventilation parameters based on feedback regarding the condition of the patient 12, e.g., measurements received from various sensors 30 or otherwise. For example, in certain embodiments, control system 20 may be configured to operate in a proportional assist ventilation (PAV) mode, in which control system 20 may execute a PAV algorithm to automatically adjust the pressure of ventilation supplied to patient 12 over time based at least on estimated measures of the patient's lung-thorax elastance, compliance, and/or resistance.

In some embodiments, control system 20 may implement “pause maneuvers” for measuring at least one of the elastance, compliance, and/or resistance associated with patient 12. A “pause maneuver” may be defined as a brief interval (e.g., 200-400 ms) during which particular valves of system 10 (e.g., one or more inspiration valves and one or more exhalation valves) are closed to create a constant volume defined at least by the patient's lungs and one or more components of system 10 (e.g., patient circuit 16). Due to the closed, constant volume, the recoil pressure in the elastic lung-thorax may equilibrate with the pressure trapped in the patient circuit 16 during the brief pause, and one or more measurements of the closed, constant volume may be taken. These measurements may then be used for calculating estimated values of the patient's lung-thorax elastance, compliance, and/or resistance, which estimated values may then be used as feedback input into the PAV algorithm such that ventilator 14 may automatically adjust the pressure of ventilation supplied to patient 12 over time based on the patient's changing elastance, compliance, and/or resistance. In alternative embodiments, pause maneuvers may comprise any other maneuver during or between assisted breaths that may be used for measuring elastance, compliance, and/or resistance of a patient. For example, in some alternative embodiments, a pause maneuver may not include closing valves to create a closed, constant volume.

A breath that includes one or more pause maneuvers may be referred to as a “pause breath,” while a breath that does not include a pause maneuver may be referred to as a “non-pause breath.” One or more pause maneuvers may be implemented at any suitable time during a pause breath. For example, in some embodiments, each pause breath includes a pause maneuver at the end of the inspiratory phase (and before the exhalation phase) of the breath.

In some embodiments, the scheduling of pause breaths (and thus pause maneuvers) may be randomized, e.g., to prevent or reduce the likelihood of patient 12 anticipating the next pause breath and consciously or subconsciously altering his or her breathing, which may be undesirable. As used herein, the term “randomized” may include partially or fully randomized or altering in any suitable manner. For example, the number of non-pause breaths between consecutive pause breaths may be frequently changing based on the output of a random number generator. As another example, pause breaths may be scheduled based on a predetermined random or pseudo-random schedule or set of numbers such that the next pause breath is not easily predictable by a patient 12. As another example, the number of non-pause breaths between consecutive pause breaths may change at least once between any three successive pause breaths.

In certain embodiments, the system may be configured such that number of non-pause breaths between consecutive pause breaths may be randomized but fall within a predefined range. For example, the number of non-pause breaths between consecutive pause breaths may be randomized between three and nine breaths (i.e., such that after each pause breath, the next pause breath will occur in four to ten breaths).

It should be understood that components of breathing assistance system 10 may include any hardware, software, firmware or other components suitable for providing ventilation assistance to patient 12, including scheduling randomized pause breaths. For example, ventilator 14 may include various processors, memory devices, sensors, user inputs, status indicators, audio devices, and/or software or other logic for providing various ventilator functions.

FIG. 2 illustrates an example gas delivery control system 20 including a proportional assist ventilation (PAV) control system 40, according to one embodiment of the disclosure. In this example embodiment, ventilator 14 is configured to operate in PAV mode (and/or any one or more other ventilation modes). PAV control system 40 may thus be operable to control ventilator 14 to provide PAV ventilation to a patient 12 according to a PAV algorithm, including scheduling randomized pause breaths having pause maneuvers for taking various measurements used for continuously or periodically updating or adjusting the PAV algorithm.

As shown in FIG. 2, PAV control system 40 may include various modules and other components for providing the various functionality associated with PAV ventilation. PAV control system 40 may include a PAV breathing controller 42, a PAV algorithm 44, a pause scheduling module 46, a pause maneuver controller 48, a pause validation module 50, a patient characteristic calculation module 52, a PAV algorithm update module 54, one or more processors 60, memory devices 62, and logic 64. In some embodiments, at least some of the modules and/or components of PAV control system 40 may comprise software modules or other logic that may be partially or fully integrated with that of other modules and/or components.

PAV breathing controller 42 may be generally operable to control ventilator 14 to deliver breathing gas to a patient 12 according to PAV algorithm 44. PAV algorithm 44 may define one or more parameters of the breathing gas to be delivered to patient 12, e.g., the pressure of the breathing gas. Inputs for PAV algorithm 44 may include values for one or more of the patient's lung-thorax elastance, compliance, and resistance, and such one or more values used in PAV algorithm 44 may be updated over time such that one or more parameters, e.g., the pressure, of gas delivered to patient 12 is adjusted over time, e.g., to account for changes in the patient's condition.

Pause scheduling module 46 may be generally operable to schedule randomized pause breaths in any suitable manner. For example, pause scheduling module 46 may include or have access to a randomizer 70 (e.g., a random number generator) and/or one or more predetermined schedules 72 of pause breaths.

Pause maneuver controller 48 may be generally operable to implement a pause maneuver during a pause breath. In some embodiments, this may include closing one or more valves of system 10 (e.g., one or more inspiration valves and one or more exhalation valves) to create a constant volume defined at least by the patient's lungs and one or more components of system 10 (e.g., patient circuit 16). Pause maneuver controller 48 may implement a pause maneuver at any suitable time during a pause breath. For example, in some embodiments, pause maneuver controller 48 may implement a pause maneuver at the end of the inspiratory phase (and before the exhalation phase) of each pause breath.

Pause validation module 50 may be generally operable to determine whether a particular pause maneuver is valid. This may include (a) determining whether one or more measurements taken during the pause maneuver are valid and/or (b) determining whether one or more patient characteristics calculated based on the one or more measurements are valid. A pause maneuver may be determined invalid for various reasons, e.g., if the patient coughs during the pause maneuver, which may disturb one or more measurements taken during the pause.

Pause validation module 50 may use any suitable techniques and/or algorithms to determine the validity of a pause maneuver. For example, pause validation module 50 may apply one or more mathematical curve-fitting techniques to determine whether strings of data obtained during a pause maneuver are valid according to some predefined criteria. As another example, pause validation module 50 may determine whether values calculated for elastance, compliance, and/or resistance based on a particular pause maneuver fall within ranges designated as valid (such ranges may be predefined or determined dynamically by the ventilator).

Patient characteristic calculation module 52 may be generally operable to calculate estimated values for at least one of the elastance, compliance, and resistance for the patient based at least on one or more measurements taken during or proximate a pause maneuver (e.g., measurements of the pressure and/or volume of the closed, constant volume) and/or one or more measurements taken during the exhalation phase of a pause breath (i.e., after the pause maneuver).

In some embodiments, patient characteristic calculation module 52 may calculate (1) estimated values for the patient's elastance and/or compliance based at least on measurement(s) taken during the pause maneuver of a particular pause breath, and (2) an estimated value for patient's resistance based at least on (a) the estimated elastance and/or compliance values and (b) measurements taken during the exhalation phase of the particular pause breath. In this manner, calculation module 52 may calculate estimated values for both (a) the elastance and/or compliance, and (b) the resistance of the patient for each pause breath. In other embodiments, patient characteristic calculation module 52 may not calculate one or more of the elastance, compliance, and/or resistance.

PAV algorithm update module 54 may be generally operable to update the values for the patient's elastance, compliance, and/or resistance used in PAV algorithm 44 with values determined by patient characteristic calculation module 52. PAV algorithm update module 54, e.g., after each pause maneuver, after a predetermined number of pause maneuvers, or according to any other schedule or pattern.

Each of the various modules and components of PAV control system 40 may include, or have access to, one or more processors 60, memory devices 62, and/or logic 64. The one more processors 60 may include any one or more types of processors, e.g., microprocessors. Memory devices 62 may include any one or more types of memory, databases, or other storage. Logic 64 may include any suitable software, algorithms, or other instructions that may be executed by one or more processors 60.

It should be understood that components of PAV control system 40 may include any hardware, software, firmware or other components suitable for providing PAV ventilation to patient 12. For example, ventilator 14 may include various processors, memory devices, sensors, user inputs, status indicators, audio devices, and/or software or other logic for providing various PAV functions.

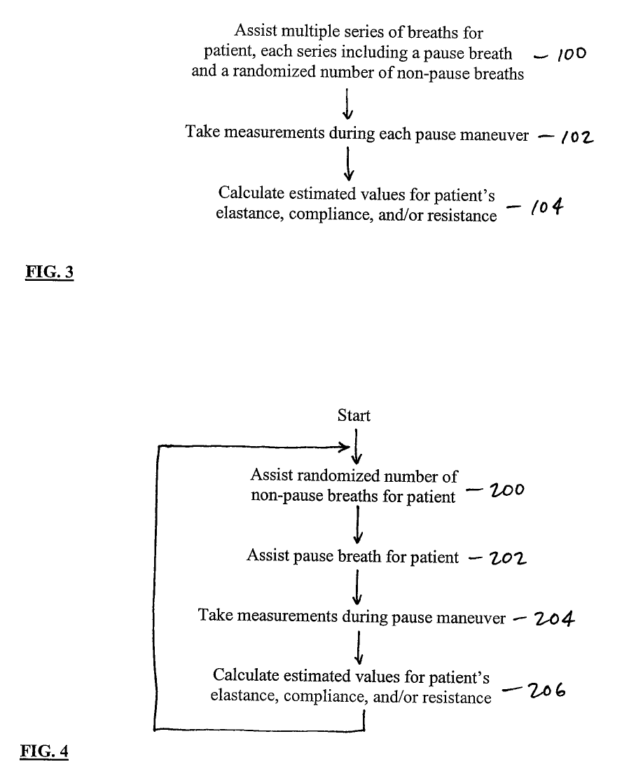

FIG. 3 illustrates an example method of using randomized pause maneuvers for determining at least one of the elastance and the compliance associated with a patient, according to one embodiment of the disclosure. At step 100, ventilator 14 assists multiple series of breaths for a patient 12. Assisting a breath for a patient may comprise providing breathing assistance to the patient during at least a portion of a breath. For example, assisting a breath for a patient may comprise providing breathing assistance to the patient during at least a portion of a patient-initiated spontaneous breath. Such breathing assistance may be provided, for example, in a PAV ventilation mode. As another example, assisting a breath for a patient may comprise providing breathing assistance to the patient during at least a portion of a ventilator-initiated or mandatory breath.

Each series of breaths may include a pause breath and a randomized number of non-pause breaths. In other words, the total number of breaths in each series may be randomized. In some embodiments, as discussed above, the randomized number of non-pause breaths in each series of breaths may fall within a predefined range, e.g., between three and nine breaths (such that the total number of breaths in each series may fall between four and ten breaths).

At step 102, during or proximate the pause maneuver of each pause breath, one or more measurements may be taken that may be used for determining values for at least one of the elastance, compliance, and resistance associated with the patient. Such measurements may include one or more measurements of the gas trapped within the closed, constant volume created during each pause maneuver (e.g., by closing appropriate valve(s)). Further, such measurements may be taken in any suitable manners, e.g., using suitable sensors 30 and/or data processing systems or devices. For example, the pressure in the closed, constant volume may be measured using a pressure sensor located at or proximate a Wye connector. As another example, the volume of the closed, constant volume may be measured by integrating a measure of the flow into the patient's lung during inspiration, which flow measure may be taken using a external flow sensor at or proximate the Wye connector. It should be understood that these techniques are only examples and that any other suitable techniques may be used.

In addition, as discussed above, in some embodiments, one or more measurements may be taken during the exhalation phase (i.e., after the pause maneuver) of the pause breath. For example, in one embodiment, the ventilator may measure the pressure at the Wye connector proximate the patient during at least a portion of the exhalation phase. Such measurements may be used for calculating the resistance associated with the patient, as discussed below.

At step 104, for each pause breath, estimated values for at least one of the elastance, compliance, and resistance associated with the patient may be calculated based at least on the one or more measurements taken for that pause breath at step 102. In some embodiments, control system 20 may execute one or more algorithms encoded in software or other logic to calculate such parameters.

In some embodiments, estimated values for the patient's elastance and/or compliance may be calculated based at least on measurements taken during the pause maneuver of a particular pause breath, and an estimated value for patient's resistance may be calculated based at least on (a) the estimated elastance and/or compliance values and (b) measurements taken during the exhalation phase of the particular pause breath. In this manner, values for (a) the elastance and/or compliance, and (b) the resistance of the patient may be determined for each pause breath.

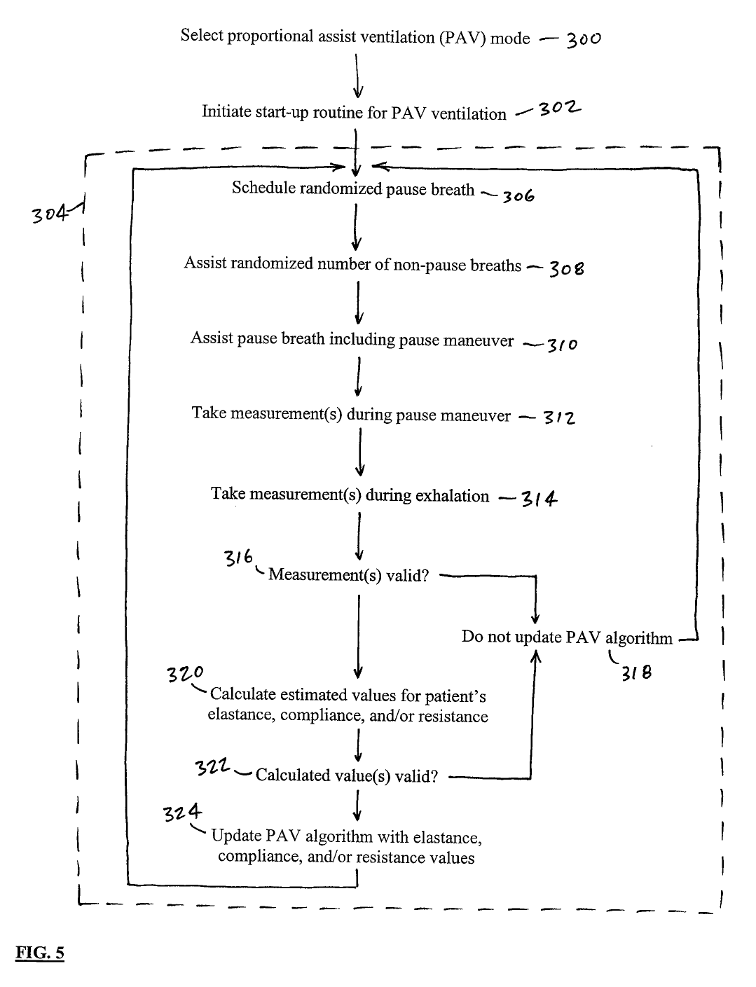

FIG. 4 illustrates another example method of using randomized pause maneuvers for determining at least one of the elastance and the compliance associated with a patient, according to one embodiment of the disclosure. At step 200, ventilator 14 assists a randomized number of non-pause breaths for a patient. The randomized number of non-pause breaths may be partially or fully randomized in any manner, e.g., as discussed herein. In some embodiments, as discussed above, the randomized number of non-pause breaths may fall within a predefined range, e.g., between three and nine non-pause breaths. In other embodiments, the randomized number of non-pause breaths may not be restricted to a predefined range.

At step 202, after the randomized number of non-pause breaths are assisted at step 200, ventilator 14 may assist a pause breath for the patient. As discussed above, a pause breath may include a pause maneuver during which one or more valves of system 10 (e.g., one or more inspiration valves and one or more exhalation valves) are closed to create a constant volume defined at least by the patient's lungs and one or more components of system 10 (e.g., patient circuit 16).

At step 204, during or proximate the pause maneuver at step 202, one or more measurements may be taken, which may be used for determining at least one of the elastance, compliance, and resistance associated with the patient, such as described above with reference to step 102 of the method of FIG. 3. For example, the pressure and/or the volume of the closed, constant volume may be measured at step 204.

In addition, in some embodiments, one or more measurements may be taken during the exhalation phase (i.e., after the pause maneuver) of the pause breath. For example, in one embodiment, the ventilator may measure the pressure at the Wye connector proximate the patient during at least a portion of the exhalation phase. Such measurements may be used for calculating the resistance associated with the patient, as discussed below.

At step 206, estimated values for at least one of the elastance, compliance, and resistance associated with the patient may be calculated based at least on the one or more measurements taken at step 204. In some embodiments, control system 20 may execute one or more algorithms encoded in software or other logic to calculate such parameters.

As discussed above with respect to FIG. 3, in some embodiments, estimated values for the patient's elastance and/or compliance may be calculated based at least on measurements taken during the pause maneuver of a particular pause breath, and an estimated value for patient's resistance may be calculated based at least on (a) the estimated elastance and/or compliance values and (b) measurements taken during the exhalation phase of the particular pause breath. In this manner, values for (a) the elastance and/or compliance, and (b) the resistance of the patient may be determined for each pause breath.

Steps 200-206 may be repeated any number of times. In some embodiments, the number of non-pause breaths assisted at step 200 may be randomized for each pass through the loop defined by steps 200-206. In other embodiments, the number of non-pause breaths assisted at step 200 may be randomized less frequently, e.g., every x times through steps 200-206. Further, the randomization may be performed in any suitable manner and at any time or times during the method. For example, in one embodiment, multiple random numbers may be determined at one time and used for multiple passes through steps 200-206. In another embodiment, the randomized number of non-pause breaths to be assisted at step 200 may be determined prior to assisting the first breath at step 200. In another embodiment, the randomized number of non-pause breaths to be assisted at step 200 may be determined after assisting one or more breaths at step 200 (e.g., after the first breath assisted at step 200).

The elastance, compliance, and/or resistance values calculated at step 206 may be used for any suitable purpose. For example, in some embodiments (e.g., in a PAV ventilation mode, as discussed below with regard to FIG. 5), such values may be used as feedback for adjusting one or more parameters (e.g., pressure) of the ventilation provided by ventilator 14.

FIG. 5 illustrates an example method of using randomized pause maneuvers for providing proportional assist ventilation (PAV), according to one embodiment of the disclosure. At step 300, an operator (e.g., a caregiver) may instruct a ventilator to provide proportional assist ventilation (PAV) for a patient, e.g., by selecting PAV ventilation from multiple types or modes of ventilation using an interface provided on the ventilator (e.g., a touch screen GUI). At step 302, the ventilator may initiate and execute a start-up routine for PAV ventilation. Such routine may include, e.g., providing minimal assistance for one or more breaths and gradually increasing the assistance provided by the ventilator based on calculated values of elastance, compliance, and/or resistance for the patient (such values may be calculated using pause maneuvers and the various techniques discussed herein).

After the start-up routine is completed, the ventilator may enter a loop, indicated as loop 304, in which the ventilator assists breaths according to a PAV algorithm, and at random intervals implements pause maneuvers during which one or more measurements are taken for calculating one or more parameters (e.g., the patient's elastance, compliance, and/or resistance) that are used as feedback for adjusting the PAV algorithm for subsequent breaths. In this manner, the PAV algorithm may be continuously or periodically updated based at least on the condition of the patient.

In an example embodiment, at each pass through loop 304, the ventilator assists a series of breaths with the last breath in each series being a pause breath. The total number of breaths (or the number of non-pause breaths) in each series of breaths may be randomized in any suitable manner. Details of this example embodiment of loop 304 are discussed below.

At step 306, pause scheduling module 46 may schedule a randomized pause maneuver for a series of breaths. For example, pause scheduling module 46 may determine the total number of breaths in the series of breaths in any randomized manner. The randomized number may be restricted within a numerical range of breaths. In some embodiments, the randomized number may be restricted within a range, e.g., between 4 and 10 breaths. In other embodiments, the randomized number may be restricted within a range of between 1 and x breaths, where x is any predetermined number greater than 1. In some embodiments, the range of breaths may be constant over time. In other embodiments, the range of breaths may be selected and/or modified by a user (e.g., a caretaker) as desired, e.g., using an interface provided on the ventilator (e.g., a touch screen GUI). In other embodiments, gas delivery control system 20 may automatically adjust or update the range of breaths over time based on any suitable input and/or according to any suitable algorithm.

At step 308, the ventilator may assist a number of non-pause breaths for the patient. In this embodiment, because the last breath in each series is a pause breath, the number of non-pause breaths in the series of breaths is one less than the randomized total number of breaths for the series determined at step 306. For example, supposing at step 306 pause scheduling module 46 determines a total of 5 breaths for the series, the ventilator may assist 4 non-pause breaths at step 308. In some embodiments in which the randomized total number of breaths is between 1 and x breaths, it may be possible that zero non-pause breaths are assisted at step 308 (if the randomized total number of breaths determined at step 306 equals 1). In an example embodiment in which the randomized total number of breaths is between 4 and 10 breaths, the number of non-pause breaths assisted at step 308 is between 3 and 9 breaths.

At step 310, after assisting the non-pause breaths at step 308, the ventilator may assist a pause breath (as the last breath of the series). As discussed above, during the pause breath, pause maneuver controller 48 may implement a pause maneuver, including closing one or more valves of system 10 (e.g., one or more inspiration valves and one or more exhalation valves) to create a constant volume defined at least by the patient's lungs and one or more components of system 10 (e.g., patient circuit 16).

At step 312, during or proximate the pause maneuver at step 310, the ventilator may take one or more measurements that may be used for determining at least one of the elastance, compliance, and resistance associated with the patient, such as described above with reference to step 102 of the method of FIG. 3. For example, the ventilator may measure the pressure and/or the volume of the closed, constant volume created during the pause maneuver.

In some embodiments, at step 314, during the exhalation phase of the pause breath (i.e., after the pause maneuver at step 310), the ventilator may take one or more measurements that may be used for determining at least the resistance associated with the patient. For example, in one embodiment, the ventilator may measure the pressure at the Wye connector proximate the patient during at least a portion of the exhalation phase.

At step 316, pause validation module 50 may determine whether the measurements taken during the pause maneuver are valid, e.g., as discussed above with respect to FIG. 2.

If the measurements are determined to be invalid, the method may then return to step 306, without updating PAV algorithm 44, to schedule a new pause maneuver for the next series of breaths, as indicated at 318.

If the measurements are determined to be valid, at step 320, patient characteristic calculation module 52 may calculate estimated values for at least one of the elastance, compliance, and resistance of the patient based at least on the one or more measurements taken at steps 312 and/or 314. In some embodiments, calculation module 52 may execute one or more algorithms encoded in logic 64 to calculate such parameters.

In some embodiments, patient characteristic calculation module 52 may (1) calculate estimated values for the patient's elastance and/or compliance based at least on measurements taken at step 312 (i.e., during the pause maneuver), and (2) calculate an estimated value for the patient's resistance based at least on (a) the calculated elastance and/or compliance values and (b) measurements taken at step 314 (i.e., during the exhalation phase of the pause breath). In this manner, patient characteristic calculation module 52 may calculate estimated values for both (a) the elastance and/or compliance, and (b) the resistance of the patient. In other embodiments, one or more of the elastance, compliance, and/or resistance may not be calculated.

At step 322, pause validation module 50 may determine whether the value(s) calculated at step 320 are valid, e.g., as discussed above with respect to FIG. 2.

If one or more of the calculated value(s) are determined to be invalid, the method may then return to step 306, without updating PAV algorithm 44, to schedule a new pause maneuver for the next series of breaths, as indicated at 318.

If calculated value(s) are determined to be valid, at step 324, PAV algorithm update module 54 may update the values for the patient's elastance, compliance, and/or resistance used in PAV algorithm 44 with the value(s) determined at step 320. The method may then return to step 306 to schedule a new pause maneuver for the next series of breaths, which breaths may be assisted according to the updated PAV algorithm 44. Loop 304 may be repeated any number of times in this manner.

Although the disclosed embodiments have been described in detail, it should be understood that various changes, substitutions and alterations can be made herein without departing from the spirit and scope of the disclosure as illustrated by the following claims.

What is claimed is:

1. A method of providing breathing assistance, comprising:
assisting a plurality of breaths for a patient using a breathing assistance system, the plurality of breaths including one or more pause breaths and one or more non-pause breaths, wherein the occurrence of pause breaths during the plurality of breaths is randomized;
during each pause breath, including a pause maneuver during which one or more valves of the breathing assistance system are closed to create a constant volume defined at least by the patient's lungs and one or more components of the breathing assistance system;
taking one or more measurements during or proximate the pause maneuver; and
determining one or more patient characteristic values based at least on the one or more measurements, the one or more patient characteristic values including values for at least one of an elastance and a compliance associated with the patient.

2. A method according to claim 1, wherein assisting a breath for the patient comprises providing breathing assistance to the patient during at least a portion of a breath.

3. A method according to claim 1, wherein assisting a breath for the patient comprises providing breathing assistance to the patient during at least a portion of a patient-initiated spontaneous breath.

4. A method according to claim 3, further comprising updating one or more parameters of breathing assistance provided to the patient for one or more subsequent breaths based at least on the one or more determined patient characteristic values.

5. A method according to claim 1, wherein the pause maneuver for each pause breath is performed at the end of an inspiration portion of the breath.

6. A method according to claim 1, further comprising, during a particular pause breath, determining a value of a resistance associated with the patient based at least on the one or more patient characteristic values determined for the particular pause breath.

7. A method according to claim 6, further comprising:
during the particular pause breath, taking one or more exhalation measurements after the pause maneuver and during an exhalation portion of the pause breath; and
wherein the value of the resistance associated with the patient is determined based at least on the one or more patient characteristic values determined for the particular pause breath and the one or more exhalation measurements.

8. A method according to claim 1, wherein taking one or more measurements during or proximate a pause maneuver comprises measuring at least one of (a) the pressure in the constant volume and (b) the volume of the constant volume.

9. A method according to claim 1, wherein measuring the volume of the constant volume comprises measuring the flow into the patient during a portion of a breath and integrating the measured flow over a time interval.

10. A method according to claim 1, wherein the plurality of breaths includes multiple series of breaths, each series of breaths including a pause breath and a randomized number of non-pause breaths.

11. A method according to claim 10, wherein the randomized number of non-pause breaths in each series of breaths is within a predetermined numerical range.

12. A method according to claim 10, wherein the randomized number of non-pause breaths in each series of breaths is within four and ten breaths.

13. A method according to claim 1, further comprising:
assisting a first randomized number of non-pause breaths for the patient;
after the first randomized number of non-pause breaths, assisting a first pause breath for the patient;
assisting a second randomized number of non-pause breaths for the patient; and
after the second randomized number of non-pause breaths, assisting a second pause breath for the patient.

14. A method according to claim 1, further comprising:
determining a first random number within a first range of numbers;
determining a number of breaths in a first series of breaths based at least on the first random number;
assisting the first series of breaths for the patient, wherein the last breath in the first series is a pause breath;
determining a second random number within a second range of numbers;
determining a number of breaths in a second series of breaths based at least on the second random number; and
assisting the second series of breaths for the patient, wherein the last breath in the second series is a pause breath.

15. A method according to claim 1, further comprising, for each of the plurality of breaths, making a randomized determination of whether that breath is a pause breath or a non-pause breath.

16. A method according to claim 1, further comprising not including the pause maneuver during each non-pause breath.

17. A method of providing breathing assistance, comprising:
assisting a plurality of series of breaths for a patient using a breathing assistance system, wherein each series of breaths includes a pause breath and a randomized number of non-pause breaths;
wherein each pause breath includes a pause maneuver during which one or more valves of the breathing assistance system are closed to create a constant volume defined at least by the patient's lungs and one or more components of the breathing assistance system; and
wherein each non-pause breath does not include the pause maneuver.

18. A method according to claim 17, wherein assisting a breath for the patient comprises providing breathing assistance to the patient during at least a portion of a breath.

19. A method according to claim 17, wherein assisting a breath for the patient comprises providing breathing assistance to the patient during at least a portion of a patient-initiated spontaneous breath.

20. A method according to claim 17, wherein the randomized number of non-pause breaths in each series of breaths is within a predetermined numerical range.

21. A method according to claim 17, wherein the randomized number of non-pause breaths in each series of breaths is between three and nine breaths.

22. A method according to claim 17, further comprising:
taking one or more measurements during or proximate each pause maneuver; and
for each pause maneuver, determining at least one of the elastance and compliance associated with the patient based at least on the one or more measurements taken during or proximate that pause maneuver.

23. A system for providing breathing assistance, comprising:
a breathing assistance controller configured to assist a plurality of breaths for a patient, the plurality of breaths including one or more pause breaths and one or more non-pause breaths;
a scheduling module configured to randomize the occurrence of pause breaths among the plurality of breaths;
a pause maneuver controller configured to include a pause maneuver in each pause breath, the pause maneuver including closing one or more valves of the breathing assistance system to create a constant volume defined at least by the patient's lungs and one or more components of the breathing assistance system;
one or more measurement devices configured to take one or more measurements during or proximate the pause maneuver; and
a patient characteristic calculation module configured to calculate one or more patient characteristic values based at least on the one or more measurements, the one or more patient characteristic values including values for at least one of an elastance and a compliance associated with the patient.

24. A system according to claim 23, wherein assisting a breath for the patient comprises providing breathing assistance to the patient during at least a portion of a breath.

25. A system according to claim 23, wherein assisting a breath for the patient comprises providing breathing assistance to the patient during at least a portion of a patient-initiated spontaneous breath.

26. A system according to claim 25, further comprising an algorithm update module configured to update one or more parameters of breathing assistance provided to the patient for one or more subsequent breaths based at least on the one or more determined patient characteristic values.

27. A system according to claim 23, wherein the patient characteristic calculation module is further configured to determine, during a particular pause breath, a value of a resistance associated with the patient based at least on the one or more patient characteristic values determined for the particular pause breath.

28. A system according to claim 27, wherein:
the one or more measurement devices are configured to take one or more exhalation measurements after the pause maneuver and during an exhalation portion of the particular pause breath; and
the patient characteristic calculation module is configured to determine the value of the resistance associated with the patient based at least on the one or more patient characteristic values determined for the particular pause breath and the one or more exhalation measurements.

29. A system according to claim 23, wherein the scheduling module configured to schedule multiple series of breaths, each series of breaths including a pause breath and a randomized number of non-pause breaths.

30. A system according to claim 29, wherein the randomized number of non-pause breaths in each series of breaths is within a predetermined numerical range.

31. A system according to claim 29, wherein the randomized number of non-pause breaths in each series of breaths is within four and ten breaths.

32. A system according to claim 23, wherein the scheduling module configured to make a randomized determination of whether that breath is a pause breath or a non-pause breath.

33. A system for providing breathing assistance, comprising:
breathing assistance control means for assisting a plurality of breaths for a patient, the plurality of breaths including one or more pause breaths and one or more non-pause breaths;
pause scheduling means for randomizing the occurrence of pause breaths among the plurality of breaths;
pause controlling means for including a pause maneuver in each pause breath, the pause maneuver including closing one or more valves of the breathing assistance system to create a constant volume defined at least by the patient's lungs and one or more components of the breathing assistance system;
measuring means for taking one or more measurements during or proximate the pause maneuver; and
patient characteristic calculation means for calculating one or more patient characteristic values based at least on the one or more measurements, the one or more patient characteristic values including values for at least one of an elastance and a compliance associated with the patient.

34. A computer-readable medium including computer-executable instructions for providing breathing assistance, comprising:
instructions for assisting a plurality of breaths for a patient using a breathing assistance system, the plurality of breaths including one or more pause breaths and one or more non-pause breaths, wherein the occurrence of pause breaths during the plurality of breaths is randomized;
instructions for including a pause maneuver in each pause breath during which one or more valves of the breathing assistance system are closed to create a constant volume defined at least by the patient's lungs and one or more components of the breathing assistance system;
instructions for taking or receiving one or more measurements during or proximate the pause maneuver; and
instructions for determining values for at least one of an elastance and a compliance associated with the patient based at least on the one or more measurements.

35. A computer-readable medium according to claim 34, further comprising instructions for randomizing the occurrence of pause breaths.
